(19) United States
(12) Patent Application Publication
(10) Pub. No.: US 2016/0313901 A1
(43) Pub. Date: Oct. 27, 2016

(54) INTERACTIVE MEDICAL SYSTEM AND METHODS

(71) Applicant: Stephen ARNOLD, Shawnigan Lake (CA)

(72) Inventor: Stephen ARNOLD, Shawnigan Lake (CA)

(21) Appl. No.: 14/692,642

(22) Filed: Apr. 21, 2015

(51) Int. Cl. G06F 3/0484; A61G 12/00

(52) U.S. Cl. CPC G06F 3/04847; A61B 19/5225; A61G 12/001; G06F 3/04842

(57) ABSTRACT

An interactive medical system and methods, involving a processor operable by way of a set of executable instructions stored in a non-transitory memory device, the set of executable instructions enabling the processor to receive, analyze, and transmit data in relation to a database, a plurality of medical equipment, and a display device, whereby an integrated medical protocol can be provided to the display device based on the analyzed data. The system is readily transportable for use in critical medical situations.

### TECHNICAL FIELD

The present disclosure relates to an interactive medical system and methods. In particular, the present disclosure relates to an interactive medical system and methods which provide information, assists with materials management and team work, and facilitates communication to medical practitioners. In particular, the present disclosure relates to an interactive medical system which assists, informs, and guides medical practitioners during clinical procedures.

### BACKGROUND

In medical emergency situations, despite extensive availability of medical information, medical practitioners are often unable to access critical information due to a lack of time. For centuries, access to medical information has been in the written form, namely embodied in text books and pictures. Existing technologies have improved access to medical information in many ways and the inclusion of audiovisual content has improved learning efficiency; however, procedures shown in such audiovisual content tend to be depicted in their entirety which renders such content inaccessible or impractical during emergencies.

For many medical practitioners, knowledge and skills are developed over repetition and exposure to specific clinical procedures. Where clinical procedures are practiced infrequently, gaps in knowledge and inefficiency result; and practitioners need assistance and visual reminders of treatment protocols. This situation is particularly true in rural and remote settings, wherein low task repetition is an unavoidable reality, and wherein inefficiency is a common result. During perinatal emergencies, time is of the essence; and inefficiency can lead to unfavorable outcomes including complications and even death.

In the related art, the use of audiovisual content has become more popular as a technique for teaching medical practitioners, and, so too, has been the virtualization of medical textbooks. Audiovisual content and virtualized medical textbooks are excellent learning tools during medical school and to teach, or refresh, a medical practitioner's knowledge, but, in their present form, audiovisual content and virtualized medical textbooks are too inefficient for use during clinical procedures.

In some related art settings, a medical practitioner's equipment is located at a plethora of locations. Current “crash carts” ameliorate some of these problems; however, current crash carts suffer from a lack of information. Primarily, related art crash carts merely contain equipment, without providing relevant medical information or, at best, providing merely generic medical information that is unrelated to, and not integrated with, the content of the related art crash cart. As such, medical practitioners who are unfamiliar with the equipment may misuse, or incorrectly use, the medical equipment. These medical practitioners, being unfamiliar with the content of the related art crash cart may also lose valuable time searching for any necessary tools.

As such, a need exists for a system and methods that address the foregoing problems of the related art. In particular, a need remains for a medical system and methods, wherein a medical practitioner can readily and quickly retrieve desired information, assimilate the desired information efficiently at a patient's bedside, as well as access and correctly use the clinical tools required for applying techniques based on the desired information.

### SUMMARY

In addressing many of the problems experienced in the related art, the present disclosure involves an interactive and integrated medical system and methods that ameliorates clinical inefficiency at multiple levels by providing at least information management, providing materials and equipment management, facilitating team work, and improving communication.

In an embodiment of the present disclosure, an interactive medical system comprises a processor operable by way of a set of executable instructions stored in a non-transitory memory device, the set of executable instructions enabling the processor to perform at least one of receive, analyze, and transmit data in relation to at least one of at least one database, a plurality of medical equipment; and at least one display device, whereby at least one integrated medical protocol is providable to the at least one display device based on the analyzed data.

In an embodiment of the present disclosure, a method of guiding a medical procedure by way of an interactive medical system, the method comprises: providing the interactive medical system, the system providing comprising providing a processor operable by way of a set of executable instructions stored in a non-transitory memory device, the set of executable instructions enabling the processor to perform at least one of receive, analyze, and transmit data in relation to at least one of at least one database, a plurality of medical equipment, and at least one display device; initiating the interactive medical system; receiving data from at least one of the plurality of medical equipment and the at least one display device; receiving data from the at least one database by way of the at least one server; analyzing data received from at least one of the plurality of medical equipment and the at least one display device with data received from the at least one database; and determining at least one integrated medical protocol based on the analyzed data.

In an embodiment of the present disclosure, a method of fabricating an interactive medical system, comprises: providing a processor operable by way of a set of executable instructions stored in a non-transitory memory device, the processor providing comprising providing the set of executable instructions for enabling the processor to perform at least one of receive, analyze, and transmit data in relation to at least one of: at least one database; a plurality of medical equipment; and at least one display device, whereby at least one integrated medical protocol is providable to the at least one display device based on the analyzed data.

Further advantages of the present disclosure will become apparent when considering the Drawing in conjunction with the below Detailed Description.

### BRIEF DESCRIPTION OF THE DRAWING

The above, and other, aspects, features, and advantages of several embodiments of the present disclosure will be more apparent from the following Detailed Description as presented in conjunction with the following several figures of the Drawing.

FIG. 1 is a block diagram illustrating an interactive medical system according to an embodiment of the present disclosure.

FIG. 2A is a flow chart illustrating a general method of guiding a medical procedure by way of an interactive medical system, according to an embodiment of the present disclosure.

FIG. 2B is a flow chart illustrating a method of providing at least one integrated medical protocol for guiding a medical procedure by way of an interactive medical system, according to an embodiment of the present disclosure.

FIG. 3 is a screen shot illustrating an initialization screen as rendered on a display device of an interactive medical system according to an embodiment of the present disclosure.

FIG. 4A is a screen shot illustrating a display associated with a selected medical practice area, such as obstetric emergencies, having at least one medical protocol related thereto, according to an embodiment of the present disclosure.

FIG. 4B is a screen shot illustrating a set of instructions for performing physical maneuvers corresponding to a medical protocol relating to respiratory distress, according to an embodiment of the present disclosure.

FIG. 5 is a diagram illustrating a front view of an interactive medical system, comprising a transporting apparatus, such as a medical cart, according to an embodiment of the present disclosure.

FIG. 6 is a diagram illustrating a back view of an interactive medical system, comprising a transporting apparatus, such as the medical cart, as shown in FIG. 5, according to an embodiment of the present disclosure.

FIG. 7 is a diagram illustrating a side view of an interactive medical system, comprising a transporting apparatus, such as medical cart, as shown in FIG. 5, according to an embodiment of the present disclosure.

FIG. 8 is a flowchart illustrating a method of fabricating an interactive medical system, according to an embodiment of the present disclosure.

The system and methods of the present disclosure will now be described with reference to the several accompanying figures of the Drawing, as follows. Corresponding reference characters indicate corresponding components throughout the several figures of the Drawing. Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood, elements that are useful or necessary in commercially feasible embodiments are often not depicted in order to facilitate a less obstructed view of these various embodiments of the present disclosure.

### DETAILED DESCRIPTION

The following description is not to be taken in a limiting sense, but is made merely for the purpose of describing the general principles of exemplary embodiments. The scope of the disclosure should be determined with reference to the Claims. Reference throughout this specification to “one embodiment,” “an embodiment,” or similar language means that a particular, feature, structure, or characteristic that is described in connection with the embodiment is included in at least one embodiment of the present disclosure. Thus, appearances of the phrases “in one embodiment,” “in an embodiment,” and similar language throughout this specification may, but do not necessarily, all refer to the same embodiment.

Further, the described features, structures, or characteristics of the present disclosure may be combined in any suitable manner in one or more embodiments. In the Detailed Description, numerous specific details are provided for a thorough understanding of embodiments of the disclosure. One skilled in the relevant art will recognize, however, that the embodiments of the present disclosure can be practiced without one or more of the specific details, or with other methods, components, materials, and so forth, and still remain encompassed by the present disclosure. In other instances, well-known structures, materials, or operations are not shown or described in detail to avoid obscuring aspects of the present disclosure.

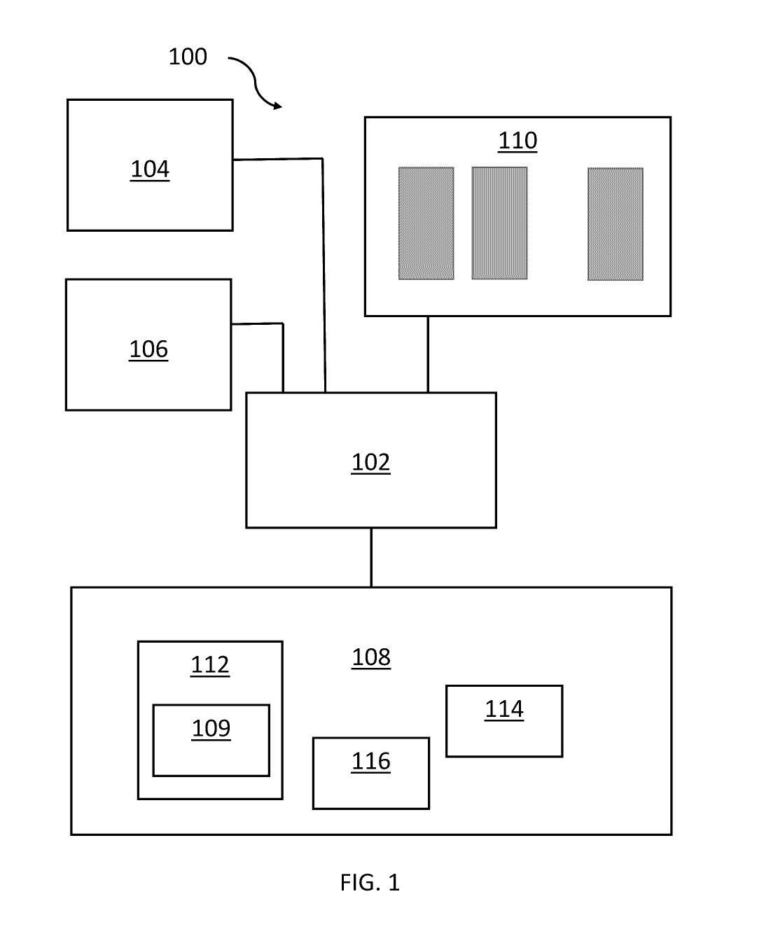

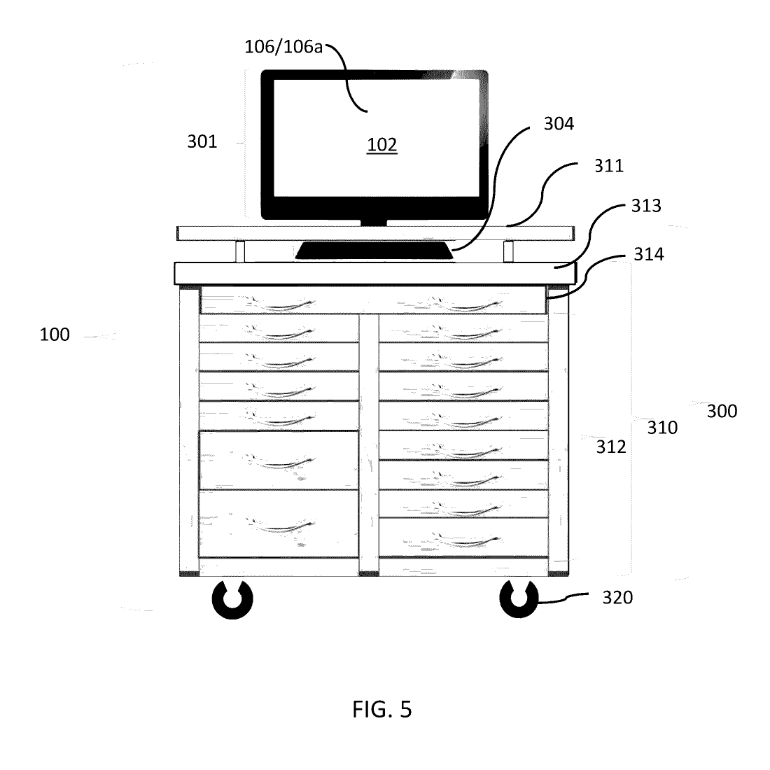

Referring to FIG. 1, this block diagram illustrates an interactive medical system 100, comprising a processor 102 operable by way of a set of executable instructions stored in a non-transitory memory device (not shown), the set of executable instructions enabling the processor 102 to perform at least one of receive, analyze, and transmit data in relation to at least one of at least one database, a plurality of medical equipment 110; and at least one display device 106, whereby at least one integrated medical protocol 160 is providable to the at least one display device 106 based on the analyzed data, according to an embodiment of the present disclosure. By example only, the interactive medical system 100 comprises a processor 102, such as a central processor unit, an input device 104, a display device 106, a plurality of medical equipment 110, and a database 108. The processor 102, for example, may be network enabled by connection to a network interface, such as Ethernet, Wi-Fi, Bluetooth®, and the like. The at least one integrated medical protocol 160 may be rendered as an image 106a by the display device 106 (See also FIG. 5).

Still referring to FIG. 1, in some embodiments, the input device 104 comprises a keyboard (not shown) and mouse (not shown) operatively connected to the processor 102. In some embodiments, the input device 104 comprises a foot mouse, such as a foot controller, operatively connected to the processor 102. In some embodiments, the input device 104 comprises a camera (not shown) configured to recognize gestures and is operatively connected to processor 102. Gestures include, but are not limited to, a head motion, a hand motion, a body motion and any combination thereof. The foot mouse (not shown) and the camera (not shown) facilitate operation of the interactive medical system 100 for at least the reason that a medical practitioner's hands are often sterilized and remaining so is desirable. These input techniques facilitate use of the interactive medical system 100 in a sterilized environment. The input device 104 further comprises an accelerometer (not shown) for facilitating motion sensing.

Still referring to FIG. 1, in some embodiments, the display device 106 comprises a monitor with a size in a range of approximately 18 inches to approximately 32 inches. In some embodiments, the display device 106 comprises a monitor with a size in a range of approximately 25 inches to approximately 29 inches. The display device 106, ranging in these sizes, facilitates contemporaneous views by a medical team, such as a plurality of medical practitioners, and facilitates coordinating medical activities. For example, in some embodiments, the medical system 100 provides at least one integrated medical treatment protocol 160 which facilitates a lead medical practitioner assigning tasks to the medical team.

Still referring to FIG. 1, in some embodiments, the display device 106 further comprises the input device 104 and are configured in a single device. In some embodiments, the single device comprises a touch screen. In these embodiments, input is facilitated by touching the touch screen of the display device 106. In some embodiments, a keyboard (not shown), such as a virtual keyboard, may be rendered on the display device 106 for facilitating text input. The processor 102 may also be configured to enable data (written or typed) onto standardized forms, via the display device 106, to be transmitted automatically to a central server (not shown), wherein the data is automatically converted into a type form and integrated into centralized tables. By example only, such data comprises nationwide resuscitation data that is collectible and analyzable by way of the processor 102.

Still referring to FIG. 1, the processor 102 is adapted to receive data from the at least one database 108, wherein the at least one database 108 is accessible from at least one of: at least one local memory device (not shown), at least one local server (not shown), at least one remote server (not shown), and at least one cloud server (not shown). In an embodiment, the at least one database 108 comprises a plurality of databases 108. Each database 108 of the plurality of databases 108 corresponds to a medical practice area 109. For example, in an embodiment, the at least one database 108 comprises information, such as data, related to at least one medical practice area 109, such as a perinatal practice area, neonatal practice area, and a cardiology practice area.

Still referring to FIG. 1, the at least one database 108 further comprises information, such as data, a plurality of medical protocols 112. Each medical protocol 112 of the plurality of medical protocols 112 comprises a set of medical instructions for performing a medical procedure or treatment, such as a set of medical techniques that are prescribed, or required, corresponding to a medical practice area 109. Each database 108 of the plurality of databases 108, optionally, further comprises videos 114 and text descriptions 116. The videos 114 and the text descriptions 116 provide additional medical information to a medical practitioner related to the medical practice area 109.

Still referring to FIG. 1, in some embodiments, the set of medical techniques in the medical protocol 112 comprises references to at least one video 114, e.g., at least one applicable videos, and at least one text description 116. References enable the processor 102 to retrieve the video 114 and the text description 116. In some embodiments, references comprise hyperlinks. In some embodiments, references comprise file paths. In some embodiments, references comprise unique keys and identifiers within the database 108.

Still referring to FIG. 1, the processor 102 is operably connected to a plurality of medical equipment 110. In some embodiments, the plurality of medical equipment 110 is customized for a specific purpose or one related medical practice area 109. In one embodiment, the plurality of medical equipment 110 is customized for use with perinatal care. In this embodiment, the plurality of medical equipment 110 comprises at least one of an ultrasound device, an electrocardiography device, a fetal heart monitor device, a medication refrigerator configured to identify at least one of the available medication(s) stored therein, the location(s) thereof, the expiratory date(s) thereof, as well as preparation and administration information thereof, a point-of-care international normalized-ratio monitoring device, a glucose monitoring device, a hemoglobin/hematocrit monitoring device, and a newborn weight scale. In another embodiment, the plurality of medical equipment 110 is customized for use in emergency life support situations. In this embodiment, the plurality of medical equipment 110 comprises at least one of a defibrillator, a suction device, a bag valve mask, advance cardiac life support medication, and pediatric equipment, such as intubation equipment and needles, an ultrasound machine, a personal-computer- (PC-) based spirometry and twelve-lead electrocardiogram (ECG) device, bedside laboratory testers, including creatinine-kinase (CK) level testers and troponin level testers, a Holter monitoring device, and a continuous blood pressure monitoring device.

Still referring to FIG. 1, in some embodiments, wherein the processor 102 is network enabled, the processor 102 is operably connectable to a plurality of medical equipment 110. In some embodiments, the plurality of medical equipment 110 comprises non-transportable medical equipment. For example, in some embodiments, at least one patient record database (not shown) is stored on a centralized database apart from the at least one database 108. In these embodiments, the processor 102 is operably connected to the at least one patient record database. The at least one patient records database is also accessible from at least one of: at least one local memory device (not shown), at least one local server (not shown), at least one remote server (not shown), and at least one cloud server (not shown). In some embodiments, the non-transportable medical equipment comprises at least one of an X-ray device, a CT scanning device, an external database, and the like. In some embodiments, the non-transportable medical equipment further comprises the at least one database 108

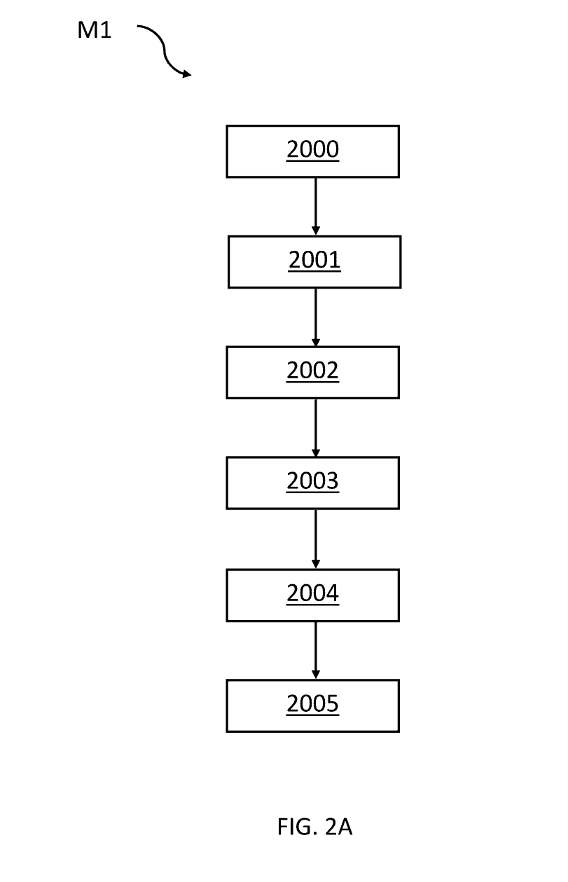

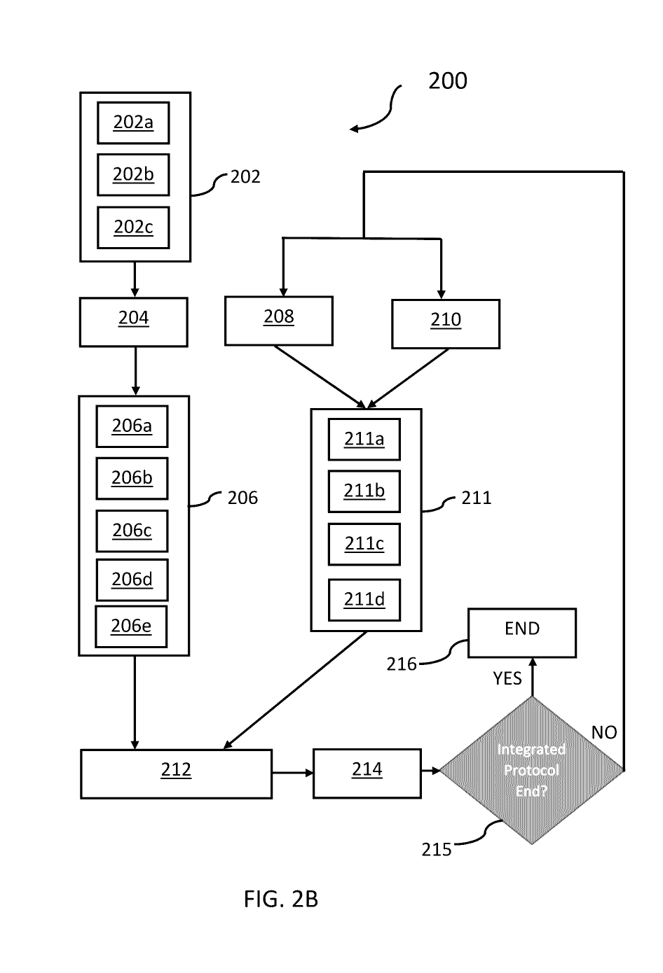

Referring to FIG. 2A, this flowchart illustrates a general method M1 of guiding a medical procedure by way of an interactive medical system 100 comprises: providing the interactive medical system 100, the system 100 providing comprising providing a processor 102 operable by way of a set of executable instructions stored in a non-transitory memory device (not shown), the set of executable instructions enabling the processor 102 to perform at least one of receive, analyze, and transmit data in relation to at least one of at least one database 108, a plurality of medical equipment 110, and at least one display device 106, as indicated by block 2000; initiating the interactive medical system 100, as indicated by block 2001; receiving data from at least one of the plurality of medical equipment 110 and the at least one display device 106, as indicated by block 2002; receiving data from the at least one database 108 by way of the at least one server (not shown), as indicated by block 2003; analyzing data received from at least one of the plurality of medical equipment 110 and the at least one display device 106 with data received from the at least one database 108, as indicated by block 2004; and determining at least one integrated medical protocol 160 based on the analyzed data, as indicated by block 2005, in accordance with an embodiment of the present disclosure.

Referring to FIG. 2B, this flow chart illustrates a method 200 of providing at least one integrated medical protocol 160 by way of an interactive medical system 100 according to an embodiment of the present disclosure. The method 200 comprises retrieving a list of medical practice areas 109 from database 108, as shown in block 202a; rendering at least one icon 109a corresponding to the at least one medical practice area 109 on display device 106, as shown in block 202b, and awaiting selection of a medical practice area from input 104, as shown in block 202c, all of which are performable as a general initial step, as indicated by block 202. In some embodiments, in the method 200, the processor 102 is operably connected to a patient records database (not shown), the processor 102 also awaits input of patient identification information from input device 104. Upon receiving input from the input device 104 by the processor 102, the method 200 further comprises retrieving the at least one medical protocol 112 corresponding to the selected medical practice area 109, as shown in block 204.

Still referring to FIG. 2B, in some embodiments, the processor 102, based on the at least one medical protocol 112 retrieved, as indicated by block 204, the method 200 further comprises identifying or determining whether the selected medical practice area 109 comprises a multi-disciplinary medical practice area, as indicated by block 206a; and determining whether an integrated medical protocol 160 is required or recommended, as shown in block 206b, all of which are performable as a general initial step, as indicated by block 206. In some embodiments, identifying or determining whether the medical practice area 109 comprises a multi-disciplinary medical practice area, as indicated by block 206a, comprises determining, e.g., by the processor 102 operating by way of a set of executable instructions storable in a non-transitory memory device (not shown). In some embodiments, the set of executable instructions comprises pre-programmed instructions for testing patient information.

Still referring to FIG. 2B, in an embodiment, the method 200 further comprises retrieving patient information, e.g., by the processor 102, as indicated by block 206c; and identifying any potential issue concerning the selected medical protocol 112 in relation to the patient information, as indicated by block 206d, all of which are also performable as a general initial step, as indicated by block 206. For example, if the selected medical practice area 109 comprises “obstetrics,” the selected medical protocol 112 comprises a “pre-mature delivery,” e.g., as a prescribed clinical procedure, and the retrieved patient information indicates that the patient suffers from hemophilia, the method 200 comprises determining, e.g., by the processor 102, based on the patient information, that a potential issue or complication exists concerning the selected medical protocol 112 in relation to the patient information, e.g., as indicated by block 206d. The method 200 further comprises retrieving, e.g., by the processor 102, at least one (additional) medical protocol 112 relating to hemophilia, as indicated by block 206e.

Still referring to FIG. 2B, in an embodiment, the method 200 further comprises at least one of: analyzing and integrating, e.g., by the processor 102 operating by way of the set of executable instructions, at least one medical protocol of the plurality of medical protocol 112, e.g., for premature delivery, with at least one additional medical protocol of the plurality of medical protocol 112, e g., for hemophilia, thereby providing an integrated medical protocol 160, as indicated by block 212; and displaying the integrated medical protocol 160 by way of the display device 106, as shown in block 214. The step of analyzing and integrating comprises utilizing at least one of smart searching and smart determining, e.g., by way of the processor 102 operating at least one of smart search engine, smart relational software, firmware, and/or hardware, and a smart command-prompt system for automatically integrating all the data and providing the best integrated medical protocol tailored to each patient in any given scenario. For instance, a set of executable instructions, relating to a medical protocol, such as for a “premature delivery,” effects a query, such as “Is there a past history of hemophilia?” to the user by way of the display device 106; and if the user provides the answer “yes,” the set of executable instructions, relating to a “premature delivery,” automatically triggers execution of another set of executable instructions relating to at least one other medical protocol.

Still referring to FIG. 2B, in some embodiments, in the method 200, displaying the integrated medical protocol 160 comprises displaying at least one selected medical protocol of the plurality of medical protocol 112, as indicated by block 202, e.g., when at least one additional medical protocol of the plurality of medical protocol 112 is not required. Accordingly, in the method 200, displaying the integrated medical protocol 160 by way of the display device 106, as shown in block 214, comprises displaying only the at least one selected medical protocol 112. The method 200 further comprises: determining, e.g., by the processor 102, whether performance of the integrated medical protocol 160 has been completed, e.g., by way of data received from the plurality of medical equipment 110, as indicated by block 215; and, if performance of the integrated medical protocol 160 has been completed, ending the performance, as indicated by block 216; and if the integrated medical protocol 160 has not been completed, at least one of: receiving data (input), e.g., by the processor 102, from the plurality of medical devices 110, as shown in block 208, and receiving additional data (input), e.g., by the processor 102, from input device 104, as shown in block 210. Alternatively, the method 200 comprises providing hyperlinks for accessing and retrieving data from at least one medical device, such as an ultrasound device, as well as other data, such as lab data, via the display device 106, wherein the data and the other data are made available via the display device 106, but the data and the other data are not automatically processed by the processor 102 by way of the set of executable instructions. In this alternative embodiment, the user as the option of self-entering the data to obtain an integrated medical protocol 160.

Still referring to FIG. 2B, in some embodiments, the method 200 comprises an iterative process, e.g., further comprising at least one of: analyzing and integrating, e.g., by the processor 102 operating by way of the set of executable instructions, at least one medical protocol from the plurality of medical protocols 112, e.g., for premature delivery, with at least one additional medical protocol from the plurality of medical protocols 112, e.g., for an additional identified medical condition (see also FIG. 1), thereby providing an integrated medical protocol 160, as indicated by block 212; and displaying the integrated medical protocol 160 by way of the display device 106, as shown in block 214, and so on and so forth. In receiving data (input), e.g., by the processor 102, from the plurality of medical equipment 110, as shown in block 208, the processor 102, implementing the integrated medical protocol 160, is adapted to expect and receive information from at least one of the plurality of medical equipment 110. For example, the processor 102, implementing the integrated medical protocol 160, prompts a medical practitioner, by way of the display device 106, to initiate hemoglobin/hematocrit monitoring, as a next procedure. Accordingly, when the medical practitioner initiates the hemoglobin/hematocrit monitoring device, the method 200 comprises retrieving data, e.g., by the processor 102, from plurality of medical equipment 110. In the method 200, comprises analyzing and integrating, e.g., by the processor 102 operating by a set of executable instructions, the hemoglobin/hematocrit information, thereby providing the integrated medical protocol 160, wherein the integrated medical protocol 160 comprises a progressive integrated medical protocol, as shown in block 212. Accordingly, in the method 200, displaying the integrated medical protocol 160 by way of the display device 106, as shown in block 214, comprises displaying the progressive integrated medical protocol.

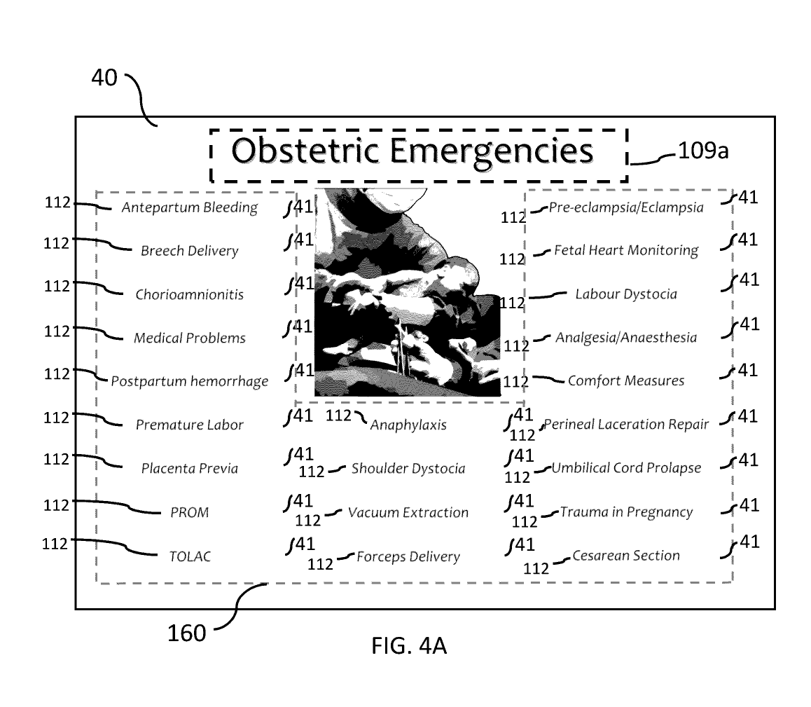

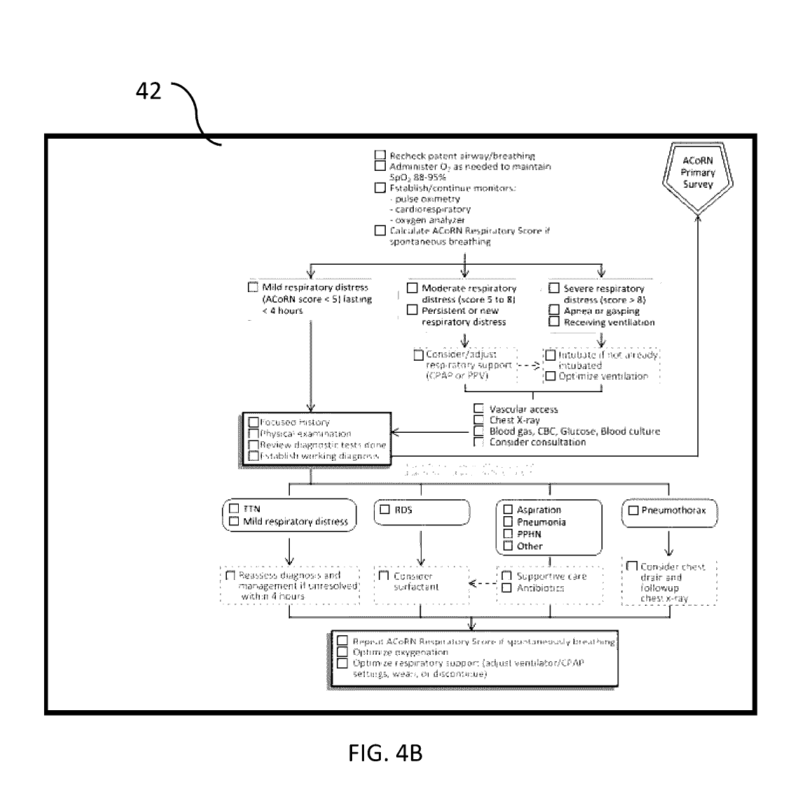

Still referring to FIG. 2B, in some embodiments, in the method 200, if the integrated medical protocol 160 has been completed, receiving additional data (input), e.g., by the processor 102, from input device 104, as shown in block 210, comprises transmitting, e.g., by the processor 102, instructions for performing physical maneuvers by the medical practitioner; and displaying the instructions for performing physical maneuvers by way of the display device 106, whereby the progressive integrated medical protocol is implementable (See also FIG. 4B). For example, the integrated medical protocol 160 comprises a set of instructions for performing a vaginal breech delivery. In one instruction of the set of instructions, the medical practitioner is required to physically deliver the baby. In one instruction of the set of instructions, the medical practitioner is provided guidance regarding the maneuvers that may be required for delivery. In some embodiments, the integrated medical protocol 160 provides instructions for physically maneuvering by at least one technique of a Mauriceau Maneuver, a forceps delivery, and an episiotomy. In this embodiment, the method 200 comprises prompting the medical practitioner to advance through the integrated medical protocol 160 by providing data (input) to the input device 104.

Still referring to FIG. 2B, in some embodiments, the method 200 further comprises at least one of transmitting a prompt, e.g., by the processor 102 to the display device 104, to the medical practitioner querying whether the practitioner would like to request further data (input), e.g., requests for medical information; as indicated by block 211a; receiving data (input) from the input device 104, comprising at least one request or displaying further medical information, as indicated by block 211b; retrieving further data or information from the at least one database 108, wherein the further data or information comprises at least one of: at least one video 114, e.g., at least one applicable video, such as an associated video, and at least one text description 116, as indicated by block 211c; and displaying (respectively streaming or rendering) the at least one video 114, e.g., at least one applicable video, such as an associated video, and at least one text description 116 on the display device 106, as indicated by block 211d, all of which are generally performed as indicated by block 211. The method 200 further comprises displaying the integrated medical protocol 160 by way of the display device 106, as shown in block 214, after displaying (respectively streaming or rendering) the at least one video 114, e.g., at least one applicable video, such as an associated video, and at least one text description 116 on the display device 106, as indicated by block 211d.

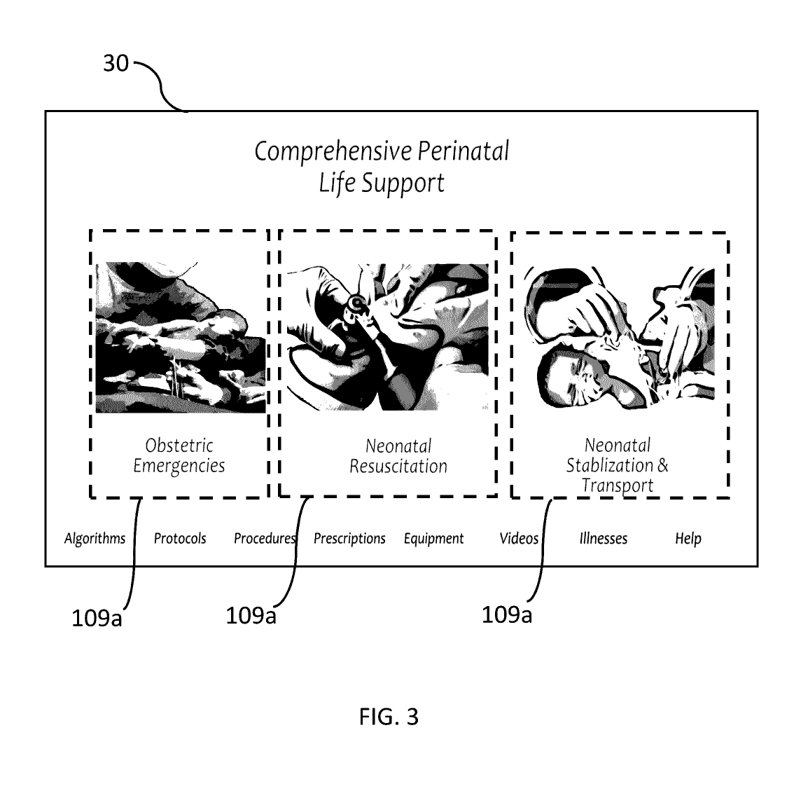

Referring to FIG. 3, this screen shot illustrates an initialization screen 30 as rendered on a display device 106 of an interactive medical system 100 according to an embodiment of the present disclosure. For instance, in the method 200, rendering the display 30 comprises rendering at least one icon 109a corresponding to the at least one medical practice area 109 on display device 106, as shown in block 202b. By example only, the display device 106 comprises a touchscreen for facilitating entry of data (input) by the medical practitioner, wherein the processor 102 is configured to receive input, e.g., data or information, such as by touching at least one icon 109a corresponding to the at least one medical practice area 109. In this embodiment, the at least one icon 109a corresponds to at least one medical practice area 109 comprising at least one of obstetric emergencies, neonatal resuscitation, neonatal stabilization and transport, Advance Life Support in Obstetrics (ALSO), Neonatal Resuscitation Program (NRP), and Acute Care of At-Risk Newborns (ACoRN), Pediatric Advanced Life Support (PALS), Advanced Life Support in Pediatrics (APLS), Acute Cardiac Life Support (ACLS), Acute Trauma Life Support (ATLS), Airway Intervention and Management in Emergencies (AIME), Fundamental Critical Care Support (FCCS), and any medical practice area involving techniques that are practiced and that complex decision-making, such as in an emergency setting.

Referring to FIG. 4A, this screen shot illustrates a display 40 associated with a selected medical practice area 109, such as obstetrics emergencies, having at least one selectable button 41 corresponding to at least one medical protocol of the plurality of medical protocol 112 and/or at least one additional medical protocol of the plurality of medical protocol 112 related thereto, according to an embodiment of the present disclosure. In the method 200, displaying the integrated medical protocol 160, e.g., comprising at least one medical protocol of the plurality of medical protocol 112 and/or at least one additional medical protocol of the plurality of medical protocol 112 by way of the display device 106, as shown in block 214, comprises rendering the display 40 on the display device 106 by way of the processor 102 transmitting data to the display device 106, according to this embodiment. By example only, the at least one selectable button 41 corresponds to at least one medical protocol of the plurality of medical protocol 112 and/or at least one additional medical protocol of the plurality of medical protocol 112, the at least one medical protocol of the plurality of medical protocol 112 and/or at least one additional medical protocol of the plurality of medical protocol 112 comprising at least one of: an antepartum bleeding protocol, a breech delivery protocol, a chorioamnionitis protocol, a medical problems protocol, a postpartum hemorrhage protocol, a premature labor protocol, a placenta previa protocol, a PROM protocol, a TOLAC protocol, an anaphylaxis protocol, a shoulder dystocia protocol, a vacuum extraction protocol, a forceps delivery protocol, a pre-eclampsia/eclampsia protocol, a fetal monitoring protocol, a labour dystocia protocol, an analgesia/anaesthesia protocol, a comfort measures protocol, a perineal laceration repair protocol, an umbilical cord prolapse protocol, a trauma-in-pregnancy protocol, and a Cesarean section protocol.

Still referring to FIG. 4A, if the integrated medical protocol 160 has been completed, receiving additional data (input), e.g., by the processor 102, from input device 104, as shown in block 210, comprises transmitting, e.g., by the processor 102, instructions for performing physical maneuvers by the medical practitioner; and displaying the instructions for performing physical maneuvers by way of the display device 106, wherein the instructions for performing the physical maneuvers are available by touching the at least one button 41, whereby the progressive integrated medical protocol is implementable. For example, the integrated medical protocol 160 comprises a set of instructions for performing a vaginal breech delivery. In one instruction of the set of instructions, the medical practitioner is provided guidance regarding the maneuvers that may be required for a delivery. In some embodiments, the integrated medical protocol 160 provides instructions for physically maneuvering by at least one technique of a Mauriceau Maneuver, a forceps delivery, and an episiotomy. In this embodiment, the method 200 comprises prompting the medical practitioner to advance through the integrated medical protocol 160 by providing data (input) to the input device 104.

Referring to FIG. 4B, this screen shot illustrates a display 42 of a set of instructions for performing physical maneuvers corresponding to a medical protocol 112 relating to respiratory distress, e.g., for a premature delivery, according to an embodiment of the present disclosure. In the method 200, displaying the integrated medical protocol 160, e.g., comprising at least one medical protocol of the plurality of medical protocol 112 and/or at least one additional medical protocol of the plurality of medical protocol 112 by way of the display device 106, as shown in block 214, further comprises rendering the display 42 on the display device 106 by way of the processor 102 receiving a signal by touching the at least one button 41 and transmitting data to the display device 106, according to this embodiment (see FIG. 2B).

Still referring to FIG. 4B, in this example, the selected medical protocol 112 comprises a set of instructions, rendered on the display 42, for a medical procedure relating to respiratory distress. The method 200 comprises analyzing and integrating, e.g., by the processor 102 operating by a set of executable instructions, the pulse oximetry, cardiorespiratory, and oxygen analyzer information, thereby providing the integrated medical protocol 160, wherein the integrated medical protocol 160 comprises a progressive integrated medical protocol, as shown in block 212 (see FIG. 2B). In receiving data (input), e.g., by the processor 102, from the plurality of medical equipment 110, as shown in block 208, the processor 102, implementing the integrated medical protocol 160, is adapted to expect and receive information from at least one of the plurality of medical equipment 110. For example, the processor 102, implementing the integrated medical protocol 160, prompts a medical practitioner, by way of the display device 106, to establish pulse oximetry, cardiorespiratory, and oxygen analyzer monitoring, as a next procedure. When the medical practitioner initiates the pulse oximetry, cardiorespiratory, and oxygen analyzer device, the method 200 comprises retrieving data, e.g., by the processor 102, from plurality of medical equipment 110. In the method 200, comprises analyzing and integrating, e.g., by the processor 102 operating by a set of executable instructions, the pulse oximetry, cardiorespiratory, and oxygen analyzer information, thereby providing the integrated medical protocol 160, wherein the integrated medical protocol 160 comprises a progressive integrated medical protocol, as shown in block 212.

Referring to FIG. 5, this diagram illustrates a front view of an interactive medical system 100, comprising a transporting apparatus 300, such as at least one of a cart and a trolley, by example only, according to an embodiment of the present disclosure. The transporting apparatus 300 comprises a body portion 310; and a shelf portion 311. The body portion 310 comprises a support surface 313; and at least one storage structure 312, such as at least one drawer, for accommodating a plurality of medical equipment 110 (See FIG. 1). The system 100 comprises a processor 102 and a display device 106, wherein the processor 100 and the display device 106 are integrated into a single device, such as an integrated processor-display device 301. The integrated processor-display device 301 further comprises and input device 304, such as at least one of a keyboard and a mouse. The integrated processor-display device 301 is configured to communicate with at least one remote server handling at least one database 108. Alternatively, the integrated processor-display device 301 is configured to store at least one database 308. In this embodiment, processor 102 is configured to execute at least one of the method M1 and the method 200, in an embodiment of the present disclosure. The system 100 further comprises at least one rotating structure 320, such as at least one wheel coupled with the transporting apparatus 300, for facilitating transport thereof. The transporting apparatus 300 further comprises at least one work surface 314, wherein the at least one work surface 314 comprises an extendable “pull-out” surface by example only.

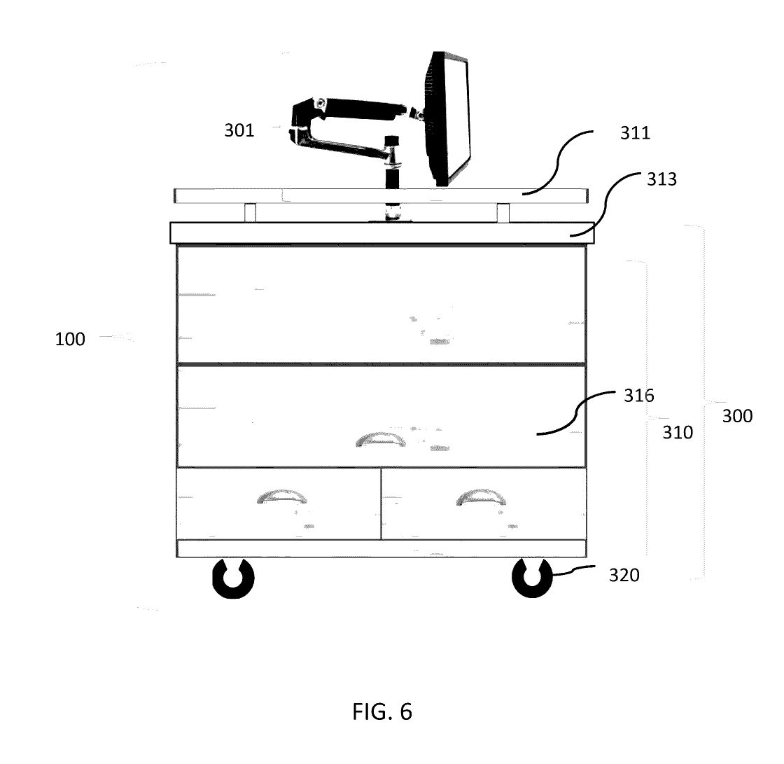

Referring to FIG. 6, this diagram illustrates a back view of an interactive medical system 100, comprising a transporting apparatus 300, such as the medical cart, as shown in FIG. 5, according to an embodiment of the present disclosure. The transporting apparatus 300 comprising at least one additional work surface 316, such as at least one retractably coupled surface, e.g., having a locking member 317, for accommodating at least one medical instrument (not shown). In this embodiment, the at least one additional work surface 314 is adapted to retractably extend in a substantially perpendicular orientation in relation to a rear surface of the transporting apparatus 300. The at least one additional work surface 316 is further adapted to rotate in a range of approximately 90 degrees in a horizontal plane and to temporally lock in place at a height approximating that of a desk height to facilitate use by a seated medical practitioner.

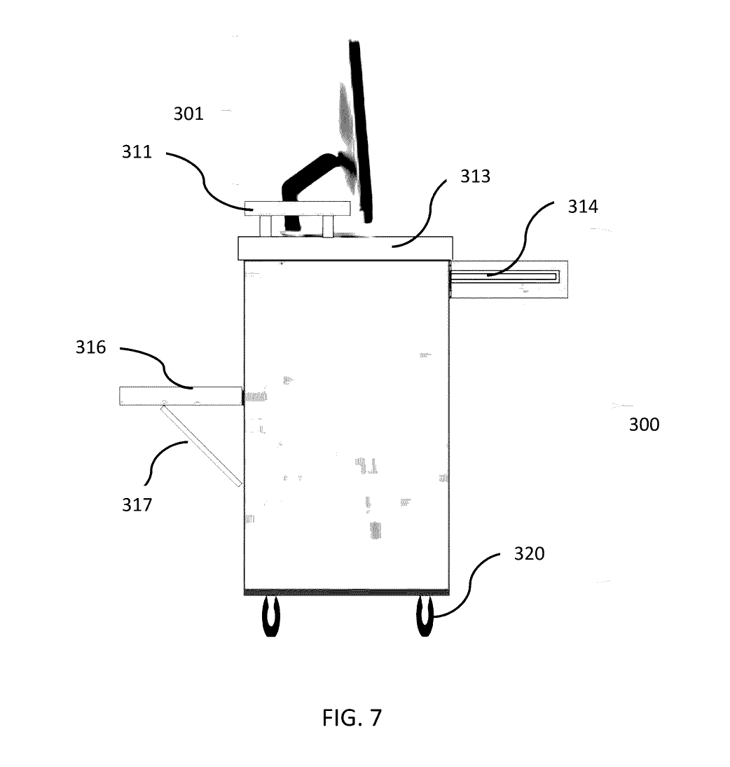

Referring to FIG. 7, this diagram illustrates a side view of an interactive medical system 100, comprising a transporting apparatus 300, such as medical cart, as shown in FIG. 5, according to an embodiment of the present disclosure. The interactive medical system 100 is customizable for perinatal care in this embodiment, wherein the database 108 is populated with protocols and medical practice areas related to perinatal care. The database 108 comprises at least one database related to a medical practice area comprising obstetrics, and a database related to the medical practice area comprising gynecology. In this embodiment, the plurality of drawers 312 accommodates a plurality of medical equipment 110, at least a portion of which is operably connected to the integrated processor-display device 301. In some embodiments, the plurality of medical equipment 110 comprises at least one of neonatal resuscitation equipment organized by age and weight, umbilical vein catheterization equipment, chest tube insertion equipment, prematurity equipment, and respiratory distress equipment. In some embodiments, the plurality of medical equipment 110 further comprises ultrasound probes operably connected to the integrated processor display device 301, an infant weight scale, a digital microscope operably connected to the integrated processor display device 301, and a refrigerator for containing the most essential medication used in the management of perinatal emergencies that require refrigeration. The most essential medication comprises at least one of Carboprost® and Ergonovine®.

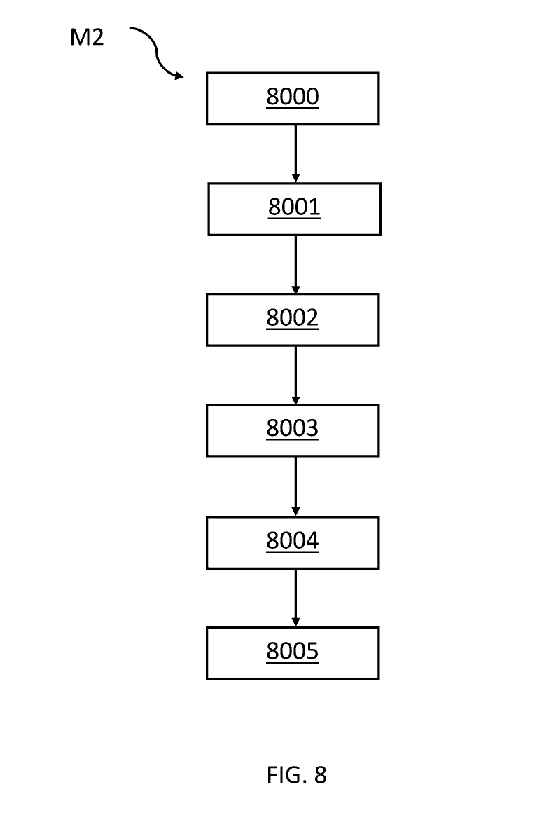

Referring to FIG. 8, this flowchart illustrates a method M2 of fabricating an interactive medical system 100, according to an embodiment of the present disclosure. The method M2 of fabricating an interactive medical system 100, comprises: providing a processor 102 operable by way of a set of executable instructions stored in a non-transitory memory device (not shown), as indicated by block 8000, the processor 102 providing comprising providing the set of executable instructions for enabling the processor 102 to perform at least one of receive, analyze, and transmit data in relation to at least one of: at least one database 108, as indicated by block 8001; a plurality of medical equipment 110, as indicated by block 8002; and at least one display device 106, as indicated by block 8003, whereby at least one integrated medical protocol 160 is providable to the at least one display device 106 based on the analyzed data. The method M2 further comprises at least one of: providing at least one of the plurality of medical equipment 110 and the at least one display device 106, as indicated by block 8004; and providing a transporting apparatus 300 for transporting at least one of the processor 102, the plurality of medical equipment 110, and the at least one display device 106, as indicated by block 8005.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become obvious to those skilled in the art, and is to be limited accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims.

Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the present disclosure.

The exemplary embodiments herein described are not intended to be exhaustive or to limit the scope of the disclosure to the precise forms disclosed. They are chosen and described to explain the principles of the disclosure and its application and practical use to allow others skilled in the art to comprehend its teachings. As will be apparent to those skilled in the art in light of the foregoing disclosure, many alterations and modifications are possible in the practice of this disclosure without departing from the scope thereof.

What is claimed is:

1. An interactive medical system, comprising: a processor operable by way of a set of executable instructions stored in a non-transitory memory device, the set of executable instructions enabling the processor to perform at least one of receive, analyze, and transmit data in relation to at least one of:
at least one database;
a plurality of medical equipment; and
at least one display device,
whereby at least one integrated medical protocol is providable to the at least one display device based on the analyzed data.

2. The system of claim 1, further comprising at least one of the plurality of medical equipment and the at least one display device.

3. The system of claim 2, further comprising means for transporting at least one of the processor, the plurality of medical equipment, and the at least one display device.

4. The system of claim 1,
wherein the at least one database is accessible from at least one of: a local device, a remote device, at least one local memory device, at least one local server, at least one remote server, and at least one cloud server, and any linked device, and
wherein the at least one database comprises a plurality of databases corresponding to a plurality of medical practice areas.

5. The system of claim 1, wherein the processor is further configured to transmit the at least one integrated medical treatment protocol to the at least one display device.

6. The system of claim 4, wherein the processor is further configured to perform at least one of:
analyze data received from at least one of the plurality of medical equipment is received by the processor; and
analyze data received from at least one of the plurality of medical equipment and the at least one display device with data received from the plurality of databases.

7. The system of claim 4, wherein the plurality of medical practice areas comprises at least two of: perinatology; neonatology, obstetrics, gynecology, endocrinology, cardiology, hematology, infectious diseases, traumatology, genetics, pharmacology, radiology, respirology, pediatrics, gastroenterology, neurology, emergency medicine, orthopedics, physiatry, any medical practice area involving at least one of physical manipulation and intervention, and any medical practice area involving techniques that are conducive to instruction via an audiovisual teaching aid.

8. The system of claim 1, wherein the plurality of medical equipment comprises at least one of: neonatal resuscitation equipment, umbilical vein catheterization equipment, chest tube insertion equipment, prematurity equipment, respiratory distress equipment, at least one ultrasound probe, at least one infant weight scale, at least one digital microscope, and at least one refrigerator.

9. The system of claim 1, wherein the transporting means comprises a medical cart.

10. The system of claim 1, further comprising:
at least one of the plurality of medical equipment and the at least one display device; and
means for transporting at least one of the processor, the plurality of medical equipment, and the at least one display device,
wherein the at least one database is accessible from at least one of: a local device, a remote device, at least one local memory device, at least one local server, at least one remote server, and at least one cloud server, and any linked device,
wherein the at least one database comprises a plurality of databases corresponding to a plurality of medical practice areas,
wherein the processor is further configured to transmit the at least one integrated medical treatment protocol to the at least one display device,
wherein the processor is further configured to perform at least one of:
analyze data received from at least one of the plurality of medical equipment is received by the processor; and
analyze data received from at least one of the plurality of medical equipment and the at least one display device with data received from the plurality of databases,
wherein the plurality of medical practice areas comprises at least two of: perinatology; neonatology, obstetrics, gynecology, endocrinology, cardiology, hematology, infectious diseases, traumatology, genetics, pharmacology, radiology, respirology, pediatrics, gastroenterology, neurology, emergency medicine, orthopedics, physiatry, any medical practice area involving at least one of physical manipulation and intervention, and any medical practice area involving techniques that are conducive to instruction via an audiovisual teaching aid,
wherein the plurality of medical equipment comprises at least one of: neonatal resuscitation equipment, umbilical vein catheterization equipment, chest tube insertion equipment, prematurity equipment, respiratory distress equipment, at least one ultrasound probe, at least one infant weight scale, at least one digital microscope, and at least one refrigerator, and
wherein the transporting means comprises a medical cart.

11. A method of providing at least one integrated medical protocol for guiding a medical procedure by way of an interactive medical system, the method comprising:
providing the interactive medical system, the system providing comprising providing a processor operable by way of a set of executable instructions stored in a non-transitory memory device, the set of executable instructions enabling the processor to perform at least one of receive, analyze, and transmit data in relation to at least one of at least one database by way of at least one server, a plurality of medical equipment, and at least one display device;
initiating the interactive medical system;
receiving data from at least one of the plurality of medical equipment and the at least one display device;
receiving data from the at least one database;
analyzing data received from at least one of the plurality of medical equipment and the at least one display device with data received from the at least one database; and
determining at least one integrated medical protocol based on the analyzed data.

12. The method of claim 11, further comprising transmitting the at least one integrated medical protocol to the at least one display device.

13. The method of claim 12, further comprising:
receiving additional data from at least one of the plurality of medical equipment and the at least one display device;
analyzing the additional data received from at least one of the plurality of medical equipment and the at least one display device with data received from the at least one database; and
determining at least one updated integrated medical protocol based on the analyzed additional data.

14. The method of claim 11, wherein the system providing comprises providing means for transporting at least one of the processor, the plurality of medical equipment, and the at least one display device.

15. The method of claim 11,
wherein receiving data from the at least one database comprises accessing data from at least one of: at least one local memory device, at least one local server, at least one remote server, and at least one cloud server, and
wherein receiving data from the at least one database comprises receiving data from a plurality of databases corresponding to a plurality of medical practice areas.

16. The method of claim 15, wherein receiving data from the plurality of databases comprises receiving data from at least two of: perinatology; neonatology, obstetrics, gynecology, endocrinology, cardiology, hematology, infectious diseases, traumatology, genetics, pharmacology, radiology, respirology, pediatrics, gastroenterology, neurology, emergency medicine, orthopedics, physiatry, any medical practice area involving at least one of physical manipulation and intervention, and any medical practice area involving techniques that are conducive to instruction via an audiovisual teaching aid.

17. The method of claim 11, wherein receiving data from the plurality of medical equipment comprises receiving data from at least one of: at least one ultrasound probe, at least one infant weight scale, and at least one digital microscope.

18. A method of fabricating an interactive medical system, comprising:
providing a processor operable by way of a set of executable instructions stored in a non-transitory memory device, the processor providing comprising providing the set of executable instructions for enabling the processor to perform at least one of receive, analyze, and transmit data in relation to at least one of:
at least one database;
a plurality of medical equipment; and
at least one display device,
whereby at least one integrated medical protocol is providable to the at least one display device based on the analyzed data.

19. The system of claim 1, further comprising providing at least one of the plurality of medical equipment and the at least one display device.

20. The system of claim 2, further comprising providing means for transporting at least one of the processor, the plurality of medical equipment, and the at least one display device.
